(19) United States
(12) Patent Application Publication
(10) Pub. No.: US 2019/0038564 A1
(43) Pub. Date: Feb. 7, 2019

(54) Carvedilol Immediate Release Formulation Having Improved Madescent

(71) Applicant: SAMJIN PHARMACEUTICAL CO., LTD., Seoul (KR)

(72) Inventors: Eui Hwan Cho, Seoul (KR); Sung-Ju Choi, Seoul (KR); Sung-Woo Lee, Seoul (KR); Hee-Jong Shin, Gyeonggi-do (KR); Min-Hyo Ki, Chungcheongnam-do (KR); Mee-Hwa Choi, Gyeonggi-do (KR); Tae-Hoon Oh, Gyeonggi-do (KR)

(21) Appl. No.: 16/075,399

(22) PCT Filed: Jan. 24, 2017
(86) PCT No.: PCT/KR2017/000811

§ 371 Date: Aug. 3, 2018

(30) Foreign Application Priority Data
Feb. 5, 2016 (KR) 10-2016-0015177

(51) Int. Cl. A61K 9/28; A61K 9/20; A61K 9/00; A61K 31/403

(52) U.S. Cl. CPC A61K 9/2866; A61K 9/284; A61K 9/282; A61K 31/403; A61K 9/2013; A61K 9/0002; A61K 9/288

(57) ABSTRACT

Disclosed is an immediate-release carvedilol formulation, which is configured such that a coating layer including a polymer, wax, fatty acid and/or fatty acid ester is formed on the surface thereof. This formulation has improved madescent, and thus can exhibit high stability, in which the initial appearance of the formulation can be maintained without cracking or breaking even under storage conditions at high relative humidity.

### TECHNICAL FIELD

The present invention relates to an immediate-release carvedilol formulation having improved madescent.

### BACKGROUND ART

Carvedilol, useful in the treatment of hypertension, is chemically named as “1-(9H-carbazol-4-yloxy)-3-[[2-(2-ketoxyphenoxy)ethyl]amino]-2-propanol”, and is represented by Chemical Formula I below.

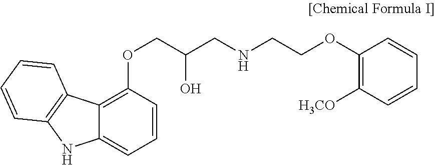 [SMILES: COc1ccccc1OCCNCC(O)COc1cccc2[nH]c3ccccc3c12]

Carvedilol functions to dilate the blood vessels through α1 and β blockage, and is used as the only third-generation β-blocker with indication for hypertension, angina pectoris, heart failure, etc. Carvedilol is very effective at decreasing blood pressure, and does not cause side effects such as edema, reflex tachycardia, dry cough and the like, which are frequently caused by other anti-hypertension drugs. It was first approved as a therapeutic agent for hypertension, especially congestive heart failure, by the U.S. Food and Drug Administration (FDA).

Carvedilol is somewhat insoluble and thus a variety of methods have been devised in order to increase the intestinal solubility thereof. For example, in currently available immediate-release carvedilol formulations, complicated processing including the preparation of a solid dispersion to solubilize carvedilol is performed, or excess disintegrant is added to the formulation.

However, in the case where carvedilol tablets are stored at high relative humidity, changes in the appearance thereof, such as side cracking or breaking thereof, may occur due to the use of excessive additives for overcoming low solubility problems and due to the properties of carvedilol drugs.

With the goal of solving the problem of madescent by carvedilol tablets, commercially available products are provided in Alu-Alu packing forms.

However, upon clinical applications, there are many cases in which carvedilol tablets are prescribed and stored in the state in which Alu-Alu packing forms are removed, and tablets having no packing may become problematic because the appearance of formulations may change, including surface swelling, side cracking, or easy tablet breaking.

Accordingly, there is urgently required an immediate-release carvedilol formulation, in which the appearance of finished products can be stably maintained without breaking even under storage conditions at high relative humidity.

### CITATION LIST

### Patent Literature

(Patent Document 1) International Patent Publication No. WO 99/52526

(Patent Document 2) International Patent Publication No. WO 2001/74357

(Patent Document 3) Korean Patent Application Publication No. 10-2005-61062

(Patent Document 4) International Patent Publication No. WO 2004/96182

(Patent Document 5) International Patent Publication No. WO 2005/51322

(Patent Document 6) Korean Patent Application Publication No. 10-2014-104341

(Patent Document 7) International Patent Publication No. WO 2002/92078

### DISCLOSURE

### Technical Problem

Accordingly, the present invention has been made keeping in mind the above problems encountered in the related art, and the present invention is intended to provide an immediate-release formulation containing carvedilol as an active ingredient, in which the formulation includes a coating layer formed on the surface thereof, and the coating layer includes at least two different kinds of components selected from the group consisting of hydroxypropyl methylcellulose, polyvinyl alcohol, a polyvinyl alcohol-polyethylene glycol copolymer, a methacrylic acid-ethyl acrylate copolymer, wax, fatty acid, and fatty acid ester.

### Technical Solution

An aspect of the present invention provides an immediate-release formulation containing carvedilol as an active ingredient, the formulation including a coating layer formed on the surface thereof, the coating layer including at least two different kinds of components selected from the group consisting of hydroxypropyl methylcellulose, polyvinyl alcohol, a polyvinyl alcohol-polyethylene glycol copolymer, a methacrylic acid-ethyl acrylate copolymer, wax, fatty acid, and fatty acid ester.

The formulation of the present invention has high stability and thus a consistent appearance even under storage conditions at high relative humidity, and enables carvedilol, which is characterized by poor solubility, to be released at a high rate.

In the present invention, carvedilol may include those available commercially or those synthesized through methods known in the art, but the present invention is not limited thereto.

In the present invention, carvedilol may be provided in any form, such as a pharmaceutically acceptable salt, an isomer, a racemate, a hydrate, and a solvate, so long as the pharmacological activity thereof is maintained uniform.

The pharmaceutically acceptable salt includes a salt derived from a pharmaceutically acceptable acid or base. In the present invention, the pharmaceutically acceptable salt indicates any organic or inorganic addition salt that is used in a concentration that is relatively non-toxic and harmless to patients and which has side effects that do not deteriorate the beneficial effects of the pharmacologically active ingredient.

The formulation of the present invention is configured such that a coating layer is formed on the surface thereof, and the coating layer functions to suppress madescent (i.e. to improve the madescent) of the formulation so that the appearance of the formulation does not change even at high relative humidity.

The coating layer may include at least two different kinds of components selected from the group consisting of hydroxypropyl methylcellulose, polyvinyl alcohol, a polyvinyl alcohol-polyethylene glycol copolymer, a methacrylic acid-ethyl acrylate copolymer, wax, fatty acid, and fatty acid ester.

Preferably, the coating layer includes at least two different kinds of components selected from the group consisting of hydroxypropyl methylcellulose, polyvinyl alcohol, a polyvinyl alcohol-polyethylene glycol copolymer, and a methacrylic acid-ethyl acrylate copolymer.

Preferably, the coating layer includes any one selected from the group consisting of hydroxypropyl methylcellulose, polyvinyl alcohol, a polyvinyl alcohol-polyethylene glycol copolymer, and a methacrylic acid-ethyl acrylate copolymer, and any one selected from the group consisting of wax, fatty acid, and fatty acid ester.

The wax may be beeswax or carnauba wax, the fatty acid may be stearic acid or palmitic acid, and the fatty acid ester may be glycerin fatty acid ester or propylene glycol fatty acid ester.

The glycerin fatty acid ester is configured such that one to three fatty acids are bound to glycerol and at least one glycerin fatty acid ester may be used, and the propylene glycol fatty acid ester is configured such that one or two fatty acids are bound to propylene glycol and at least one propylene glycol fatty acid ester may be used.

More preferably, the coating layer includes polyvinyl alcohol and propylene glycol fatty acid ester; polyvinyl alcohol and glycerin fatty acid ester; polyvinyl alcohol and a polyvinyl alcohol-polyethylene glycol copolymer; polyvinyl alcohol and a methacrylic acid-ethyl acrylate copolymer; hydroxypropyl methylcellulose and stearic acid; hydroxypropyl methylcellulose and wax.

The coating layer may further include a plasticizer, a shading agent, a colorant, a pharmaceutically acceptable excipient, and mixtures thereof.

For example, the coating layer may further include at least one selected from the group consisting of microcrystalline cellulose, sodium lauryl sulfate, polyethylene glycol 6000, silicon dioxide, titanium oxide, and talc.

The coating layer of the present invention may be applied on a formulation using a process known in the art, and, for example, components contained in the coating layer may be prepared in the form of a solution or a suspension and then applied on the formulation. As necessary, a stack of two or more coating layers may be applied.

The coating layer is used in an amount of 0.1 to 20 wt %, and preferably 1 to 10 wt %, based on the total weight of the formulation.

In the formulation of the present invention, the coating layer has an effect in preventing and inhibiting madescent.

In a specific Example, an uncoated tablet containing carvedilol as an active ingredient was manufactured and then coated with a coating dispersion comprising components for a coating layer of the present invention, which are mixed, thereby obtaining an immediate-release carvedilol-coated tablet having improved madescent.

In a specific Test Example, coated tablets (Examples 1 to 8), uncoated tablets (Comparative Examples 1 to 2) and a coated tablet having another coating layer in lieu of the coating layer of the present invention (Comparative Example 3) were stored under accelerated storage conditions, and the appearances thereof were compared after 6 hr, 5 days and 27 days. Consequently, the coated tablets of the Examples exhibited superior stability, in which the appearances thereof were not changed, but the uncoated tablets or the coated tablet having the other coating layer in lieu of the coating layer of the present invention showed surface swelling and cracking over time, undesirably breaking the formulations.

The formulation of the present invention is able to release 65% or more of carvedilol within 30 min in a pH 4.5 citric acid buffer solution.

In a specific Test Example, the coated tablets of the Examples were subjected to a dissolution test in accordance with the General Test Methods in the Korean Pharmacopoeia, as a consequence of which the coated tablets of all the examples according to the present invention can be found to rapidly release carvedilol (Table 3).

The formulation of the present invention may include, in addition to the carvedilol active ingredient, at least one additive selected from the group consisting of a diluent, a disintegrant, a binder, and a lubricant, as necessary.

The diluent may be selected from among white sugar, D-mannitol, lactose and starch, the disintegrant may be selected from among croscarmellose sodium, crospovidone or sodium starch glycolate, the binder may be selected from among hydroxypropyl methylcellulose (HPMC), hydroxypropyl cellulose (HPC), polyvinyl alcohol (PVA) and povidone (PVP), and the lubricant may be selected from among talc, silicon dioxide, stearic acid, magnesium stearate, and sodium stearyl fumarate.

The immediate-release carvedilol formulation of the present invention may be provided in the form of a tablet or a capsule.

### Advantageous Effects

According to the present invention, an immediate-release carvedilol formulation is configured such that a coating layer for preventing madescent is formed on the surface thereof, whereby the appearance of the formulation is made consistent without cracking or breaking, and moreover, the release of carvedilol is not hindered by the coating layer, thus maintaining excellent immediate-release characteristics.

### DESCRIPTION OF DRAWING

FIG. 1 shows the appearances of Examples and Comparative Examples under accelerated storage conditions in Test Example 2.

### MODE FOR INVENTION

A better understanding of the present invention may be obtained through the following examples which are set forth to illustrate, but are not to be construed as limiting the present invention.

### Comparative Examples 1 to 3

Carvedilol, crushed white sugar, mannitol, lactose hydrate, crospovidone and light anhydrous silicic acid were mixed in the amounts shown in Table 1 below and granulated with a povidone solution obtained by dissolving povidone in purified water. The granulated product was dried, tableted, mixed with crospovidone, light anhydrous silicic acid and magnesium stearate, and tableted (pressed), thus manufacturing the uncoated tablets of Comparative Examples 1 to 3. The uncoated tablet of Comparative Example 3 was additionally coated with a coating dispersion comprising hydroxypropyl methylcellulose, titanium oxide and polyethylene glycol 400 in the amounts shown in Table 1 below, thus forming a coated tablet.

TABLE 1

(unit: mg)

| Component | C. Ex. 1 | C. Ex. 2 | C. Ex. 3 |
|---|---|---|---|
| Carvedilol | 12.5 | 12.5 | 12.5 |
| Crushed white sugar | 13.0 | — | 13.0 |
| Mannitol | — | 13.0 | — |
| Lactose hydrate | 64.5 | 64.5 | 64.5 |
| Crospovidone | 6.0 | 6.0 | 6.0 |
| Light anhydrous silicic acid | 2.0 | 2.0 | 2.0 |
| Povidone | 0.5 | 0.5 | 0.5 |
| Magnesium stearate | 1.5 | 1.5 | 1.5 |
| Hydroxypropyl methylcellulose/ | — | — | 3 |
| titanium oxide/polyethylene | | | |
| glycol 400 (ratio: 62.5/31.2/6.3) | | | |
| Total weight | 100 | 100 | 103 |

### Examples 1 to 8

Coated tablets of Examples 1 to 8 were formulated by coating the uncoated tablets of Comparative Examples 1 and 2 with coating dispersions comprising the components shown in Table 2 below in the amounts corresponding to film coating rates (%).

TABLE 2
(unit: mg)

| | | Example | | | | | | | |
|---|---|---|---|---|---|---|---|---|---|
| | | 1 | 2 | 3 | 4 | 5 | 6 | 7 | 8 |
| | | Uncoated tablet | | | | | | | |
| Component | | C. Ex. 1 | | | | | | C. Ex. 2 | |
| Coating | Hydroxypropyl methylcellulose | — | — | — | — | 65 | 55 | — | — |
| dispersion | Polyvinyl alcohol | 48 | 48 | 37.8 | 42 | — | — | 48 | 37.8 |
| | Polyvinyl alcohol-polyethylene | — | — | 62 | — | — | — | — | 62 |
| | glycol copolymer | | | | | | | | |
| | Methacrylic acid- | — | — | — | 28 | — | — | — | — |
| | ethyl acrylate copolymer | | | | | | | | |
| | Stearic acid | — | — | — | — | 10 | — | — | — |
| | Glycerin fatty acid ester | — | 18 | — | — | — | — | 18 | — |
| | Wax | — | — | — | — | — | 17 | — | — |
| | Propylene glycol fatty acid ester | 18 | — | — | — | — | — | — | — |
| | Microcrystalline cellulose | — | — | — | — | 10 | — | — | — |
| | Sodium lauryl sulfate | — | 0.2 | — | 0.2 | — | | 0.2 | — |
| | Polyethylene glycol 6000 | 0.2 | — | — | 8 | — | 8 | — | — |
| | Silicon dioxide | — | — | 0.2 | — | — | — | — | 0.2 |
| | Titanium oxide | 13.8 | 13.8 | — | 7.8 | 15 | 15 | 13.8 | — |
| | Talc | 20 | 20 | — | 14 | — | 5 | 20 | — |
| | Total weight of coating dispersion | 100 | 100 | 100 | 100 | 100 | 100 | 100 | 100 |

| | | | | | | | | |
|---|---|---|---|---|---|---|---|---|
| Film coating rate of coating dispersion (%) | 4 | 6 | 4 | 8 | 4 | 5 | 4 | 10 |
| Total weight of coated tablet | 104 | 106 | 104 | 108 | 104 | 105 | 104 | 110 |

### Test Example 1

The coated tablets of Examples 1 to 8, the uncoated tablets of Comparative Examples 1 and 2, and the coated tablet of Comparative Example 3 were subjected to a dissolution test at 90 rotations per min in accordance with Method 2 of the Dissolution Test of the General Test Methods in the Korean Pharmacopoeia. 30 min after initiation of the test, the release rate of carvedilol was measured at a wavelength of 285 nm using a UV absorption spectrometer. The results are shown in Table 3 below. The test solution was 1000 mL of a pH 4.5 citric acid buffer solution.
Preparation of citric acid buffer solution: 138 g of citric acid and 57.5 g of sodium hydroxide were dissolved in 8 L of purified water, and the pH of the resulting solution was adjusted to pH 4.5 with about 32.5 mL of 25% hydrochloric acid, and the amount thereof was adjusted to 10 L using purified water.

TABLE 3

| | Release rate of carvedilol after 30 min (%) |
|---|---|
| Ex. 1 | 93.73 |
| Ex. 2 | 92.15 |
| Ex. 3 | 93.28 |
| Ex. 4 | 91.98 |
| Ex. 5 | 91.96 |
| Ex. 6 | 90.47 |
| Ex. 7 | 90.54 |
| Ex. 8 | 88.26 |
| C. Ex. 1 | 94.67 |
| C. Ex. 2 | 92.52 |
| C. Ex. 3 | 92.85 |

As is apparent from Table 3, all of the coated tablets of Examples 1 to 8, the uncoated tablets of Comparative Examples and 2, and the coated tablet of Comparative Example 3 exhibited a carvedilol release rate of 65% or more within a predetermined time (30 min). Thereby, all of the coated tablets of Examples 1 to 8, the uncoated tablets of Comparative Examples 1 and 2, and the coated tablet of Comparative Example 3 were found to be formulations able to immediately release carvedilol.

### Test Example 2

The coated tablets of Examples 1 to 8, the uncoated tablets of Comparative Examples 1 and 2, and the coated tablet of Comparative Example 3, none of which were packed, were stored under the following storage conditions, and the appearances thereof were observed.
Storage conditions: accelerated storage conditions (40±2° C./relative humidity 75±5%)

TABLE 4

| | Initial | 6 hr | 5 days | 27 days |
|---|---|---|---|---|
| Ex. 1 | Good | Good | Good | Good |
| Ex. 2 | Good | Good | Good | Good |
| Ex. 3 | Good | Good | Good | Good |
| Ex. 4 | Good | Good | Good | Good |
| Ex. 5 | Good | Good | Good | Good |
| Ex. 6 | Good | Good | Good | Good |
| Ex. 7 | Good | Good | Good | Good |
| Ex. 8 | Good | Good | Good | Good |
| C. Ex. 1 | Good | Surface swell- | Surface swell- | Surface swell- |
| | | ing, cracking, | ing, cracking, | ing, cracking, |
| | | easy breaking | easy breaking | easy breaking |
| C. Ex. 2 | Good | Surface swell- | Surface swell- | Surface swell- |
| | | ing, cracking, | ing, cracking, | ing, cracking, |
| | | easy breaking | easy breaking | easy breaking |
| C. Ex. 3 | Good | Good | Surface swell- | Surface swell- |
| | | | ing, cracking, | ing, cracking, |
| | | | easy breaking | easy breaking |

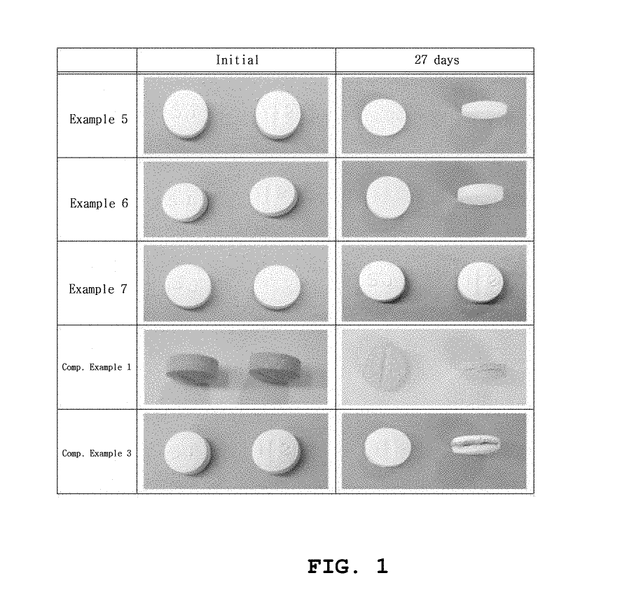

As shown in Table 4, when the uncoated tablets of Comparative Examples 1 and 2 were stored at high relative humidity, changes in the appearance thereof, such as surface swelling and side cracking, occurred during storage for 6 hr. As is apparent from Table 4 and FIG. 1, the coated tablet of Comparative Example 3 maintained its initial appearance for 6 hr, but the initial appearance thereof was not maintained, but side cracking occurred by the 5 days in storage.

In contrast, as shown in Table 4 and FIG. 1, the coated tablets of Examples 1 to 8 were found to maintain their initial appearances, without changes in appearance thereof, not only after storage for 5 days but also after storage for 27 days.

1. An immediate-release formulation containing carvedilol as an active ingredient, wherein the formulation includes a coating layer formed on a surface thereof, and the coating layer includes at least one kind of component selected from the group consisting of polyvinyl alcohol, a polyvinyl alcohol-polyethylene glycol copolymer, a methacrylic acid-ethyl acrylate copolymer, wax, fatty acid, and fatty acid ester.

2. An immediate-release formulation containing carvedilol as an active ingredient, wherein the formulation includes a coating layer formed on a surface thereof, and the coating layer includes at least two different kinds of components selected from the group consisting of hydroxypropyl methylcellulose, polyvinyl alcohol, a polyvinyl alcohol-polyethylene glycol copolymer, a methacrylic acid-ethyl acrylate copolymer, wax, fatty acid, and fatty acid ester.

3. The formulation of claim 2, wherein the coating layer includes any one selected from among hydroxypropyl methylcellulose and polyvinyl alcohol; and any one selected from among a polyvinyl alcohol-polyethylene glycol copolymer, a methacrylic acid-ethyl acrylate copolymer, wax, fatty acid and fatty acid ester.

4. The formulation of claim 2, wherein the coating layer includes polyvinyl alcohol and fatty acid ester; polyvinyl alcohol and a polyvinyl alcohol-polyethylene glycol copolymer; polyvinyl alcohol and a methacrylic acid-ethyl acrylate copolymer; hydroxypropyl methylcellulose and fatty acid; or hydroxypropyl methylcellulose and wax.

5. The formulation of claim 2, wherein the coating layer is used in an amount of 1 to 10 wt % based on a total weight of the formulation.

6. The formulation of claim 2, wherein the formulation includes at least one additive selected from the group consisting of a diluent, a disintegrant, a binder, and a lubricant.

7. The formulation of claim 2, wherein the formulation releases 65% or more of carvedilol within 30 min in a pH 4.5 citric acid buffer solution.

8. The formulation of claim 2, wherein the formulation prevents madescent.
